(19) United States
(12) Patent Application Publication
(10) Pub. No.: US 2026/0158207 A1
(43) Pub. Date: Jun. 11, 2026

(54) MEDICAL DEVICES INCLUDING SEALS AND RELATED SYSTEMS AND METHODS

(71) Applicants: Boston Scientific Scimed, Inc., Maple Grove, MN (US); Boston Scientific Medical Device Limited, Galway (IE)

(72) Inventors: Nitesh Ghananil BAVISKAR, Kalyan West (IN); Benjamin CLEVELAND, Bellingham, MA (US); Snehal PATIL, Pune (IN); Shrikant Vasant RAUT, Pune (IN); John SUBASIC, Sutton, MA (US)

(73) Assignees: Boston Scientific Scimed, Inc., Maple Grove, MN (US); Boston Scientific Medical Device Limited, Galway (IE)

(21) Appl. No.: 19/405,810

(22) Filed: Dec. 2, 2025

Related U.S. Application Data
(60) Provisional application No. 63/728,285, filed on Dec. 5, 2024.

(51) Int. Cl. A61M 1/00

(52) U.S. Cl. CPC A61M 1/84

(57) ABSTRACT

Medical devices and related methods are described. The medical device may include a handle having a proximal end that includes a seal. The seal may include a first portion and a second portion. The first portion may define a proximal opening configured to receive a shaft of a medical instrument, at least part of the first portion proximate the proximal opening being a flexible membrane. The second portion may be integral with or coupled to the first portion. The flexible membrane may be movable relative to the second portion to form and maintain a fluid-tight seal with the shaft.

### CROSS-REFERENCE TO RELATED APPLICATIONS

This application claims the benefit of priority from U.S. Provisional Application No. 63/728,285, filed on Dec. 5, 2024, which is incorporated by reference herein in its entirety.

### TECHNICAL FIELD

Aspects of the present disclosure relate generally to medical devices and related systems and methods useful in aspiration. More specifically, the present disclosure relates to medical devices having a seal, e.g., to facilitate aspiration during medical procedures.

### BACKGROUND

During a medical procedure, medical professionals may use an aspiration sheath coupled to a vacuum source to provide suction during the medical procedure. The aspiration sheath may include a seal configured to form a fluid-tight seal against a medical device, for example, to maintain a vacuum pressure within the aspiration sheath. During the medical procedure, the fluid-tight seal against the medical device may be broken or disrupted, resulting in a loss of pressure within the aspiration sheath. A loss of pressure within the aspiration sheath may present challenges while performing a procedure.

### SUMMARY

The present disclosure relates to, among other things, medical devices and assemblies for providing a liquid-tight seal against an insertion portion. Each aspect disclosed herein may include one or more features described in connection with any other disclosed aspect.

According to some aspects, the present disclosure includes a medical device that includes a handle having a proximal end that includes a seal. The seal may include a first portion and a second portion. The first portion may define a proximal opening configured to receive a shaft of a medical instrument, at least part of the first portion proximate the proximal opening being a flexible membrane. The second portion may be integral with or coupled to the first portion. The flexible membrane may be movable to the second portion to form and maintain a fluid-tight seal with the shaft.

Any of the medical devices disclosed herein may include any or all of the following features in any combination. The flexible membrane of the seal may be movable relative to the second portion in a radial direction or a longitudinal direction. The proximal opening of the seal may be distal to a proximal-most end of the seal, or may be proximal to a proximal-most end of the seal. The proximal opening of the seal may be tapered.

The flexible membrane of the seal may include at least one fold. The seal may include a cavity in fluid communication with the proximal opening. A wall of the first portion that defines the proximal opening of the seal includes at least one annular groove. The seal may include an internal wall between the first portion and the second portion. The internal wall may include an internal opening aligned with the proximal opening. A wall of the seal may include a protrusion extending towards an internal cavity of the seal.

The second portion of the seal may be integral with the first portion of the seal. A wall of the first portion of the seal may vary in thickness. The first portion of the seal may have a tapered outer surface. The seal may be fixedly coupled to the handle.

The present disclosure also includes a medical device comprising a handle having a proximal end that includes a seal. The seal may include a first portion defining a proximal opening configured to receive a shaft of a medical instrument. At least part of the first portion proximate the proximal opening may be a flexible membrane. The seal may further include a second portion integral with or coupled to the first portion. The flexible membrane may be movable relative to the second portion to form and maintain a fluid-tight seal with the shaft.

Any of the medical devices disclosed herein may include any or all of the following features in any combination. The flexible membrane of the seal may be movable relative to the second portion in a radial direction or in a longitudinal direction. The proximal opening of the seal may be distal to a proximal-most end of the seal, or may be proximal to a proximal-most end of the seal. The proximal opening of the seal may be tapered. The flexible membrane of the seal may include at least one fold.

The seal may include a cavity in fluid communication with the proximal opening. A wall of the first portion that defines the proximal opening of the seal may include at least one annular groove. The seal may include an internal wall between the first portion and the second portion. The internal wall may include an internal opening aligned with the proximal opening. A wall of the seal may include a protrusion extending towards an internal cavity of the seal. The second portion of the seal may be integral with the first portion of the seal. A wall of the first portion of the seal may vary in thickness. The first portion of the seal may have a tapered outer surface. The seal may be fixedly coupled to the handle.

The present disclosure also includes a medical device including a seal comprising a biocompatible polymer. The seal may include a first portion defining a proximal opening configured to receive a shaft of a medical instrument. At least part of the first portion proximate the proximal opening may be a flexible membrane. A second portion may be coupled to the first portion. Relative rotation between the first portion and the second portion may move the flexible membrane to thereby increase or decrease a size of the proximal opening, e.g., to form and maintain a fluid-tight seal with the shaft of the medical instrument.

Any of the medical devices disclosed herein may include any or all of the following features in any combination. The flexible membrane of the seal may include a plurality of slits extending radially outward relative to a longitudinal axis of the seal. The seal may be integrally formed with or fixedly coupled to a handle of the medical device.

### BRIEF DESCRIPTION OF THE FIGURES

The accompanying drawings, which are incorporated in and constitute a part of this application, illustrate aspects of this disclosure and together with the description, serve to explain the principles of this disclosure. The drawings show exemplary medical devices useful for providing and controlling suction during a medical procedure.

FIG. 1 illustrates a schematic of an exemplary medical system, according to aspects of this disclosure.

FIGS. 2A-2B, 3A-3B, 4A-4B, 5, 6A-6D, 7A-7B, and 8A-8B depict exemplary seals useful for medical devices and systems, according to aspects of the present disclosure.

### DETAILED DESCRIPTION

Both the foregoing general description and the following detailed description are exemplary and explanatory only and are not restrictive of the features, as claimed. As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range. The terms “proximal” and “distal” are used herein to refer to the relative positions of the components of exemplary medical devices. As used herein, “proximal” refers to a position relatively closer to the exterior of the body or closer to an operator using the medical device (see proximal “P” and distal “D” directional arrows in the figures). In contrast, “distal” refers to a position relatively further away from the operator using the medical device, or closer to the interior of the body.

Aspects of the present disclosure are described herein with reference to exemplary medical devices and systems that may be useful for facilitating suction during medical procedures. The medical system may include a medical device that includes a seal configured to receive at least a portion of an instrument. For example, the medical device may be or include an aspiration sheath. In some examples, the medical device includes a handle and a sheath, the medical device configured to receive a shaft of a medical instrument (e.g., a shaft of a scope or other medical instrument). The medical device may be configured to receive the shaft via a proximal opening of a seal of the handle of the medical device. The shaft may extend through, and be slidable relative to, the seal of the medical device. The medical device and/or medical instrument may be used to provide suction and/or deliver irrigation at or proximate a target site of a subject. The seal of the medical device may be configured to allow a user to manipulate the medical instrument within the medical device, e.g., without substantially affecting pressure of the medical device. For example, the seal may include features to permit lateral movement, longitudinal movement, and rotation of the medical instrument relative to the medical device without affecting suction pressure of the medical device.

References to a particular type of procedure, such as a urology procedure; a medical device or medical instrument, such as an aspiration device or scope; and/or an organ, such as a kidney, are provided for convenience and not intended to limit this disclosure. Accordingly, analogous medical devices or systems may utilize one or more aspects of exemplary medical devices, systems, and methods described herein.

Reference is now made in detail to examples to help illustrate aspects of the present disclosure through the accompanying drawings. Wherever possible, the same reference numbers will be used throughout the drawings to refer to the same or like parts.

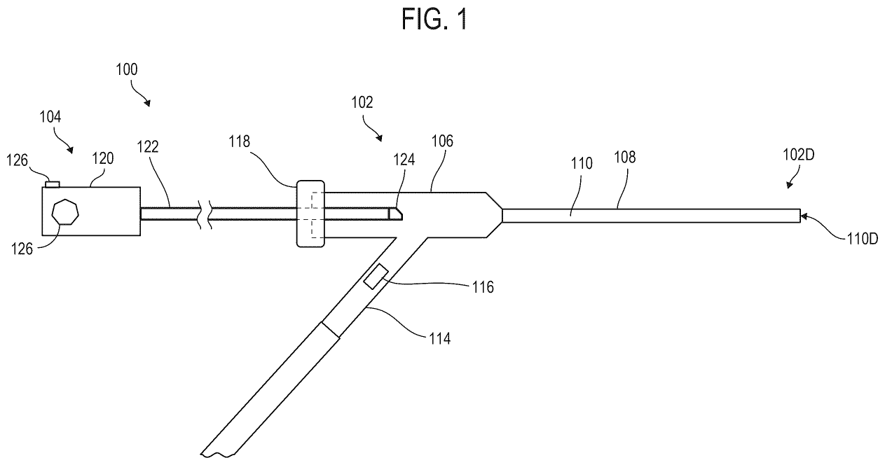

FIG. 1 illustrates an exemplary medical system 100, including a medical device 102 and a medical instrument 104. Medical device 102 may be an aspiration sheath, for example. Medical instrument 104 may be a scope, for example. Medical instrument 104 may include a handle 120 coupled to shaft 122, handle 120 having one or more actuators 126 configured to articulate and/or actuate aspects of shaft 122 and/or distal tip 124 of shaft 122. According to some aspects of this disclosure, medical device 102 may be configured to allow a user (e.g., a medical professional) to deliver medical device 102 and medical instrument 104 to access internal areas of a subject's body, e.g., to perform medical diagnoses and/or treatments on the subject. A negative pressure (e.g., vacuum) may be applied to the target site using medical device 102. The negative pressure applied via medical device 102 may allow for the removal of material(s) such as fluids and/or bodies (e.g., stones, tissue, foreign bodies, etc.) from the subject.

Medical device 102 may include a handle 106 and a sheath 108 extending distally from handle 106. Sheath 108 may include a lumen 110 in fluid communication with a port 114 of handle 106. The lumen 110 may have a distal opening 110D at or adjacent to a distal end 102D of sheath 108. Port 114 may be coupled to a vacuum source, e.g., to provide suction through lumen 110. Port 114 may include an opening 116 serving as a vent to adjust pressure in medical device 102. Opening 116 may be covered or uncovered, for example, to increase or decrease pressure, respectively, within medical device 102 during the application of negative pressure via port 114.

Handle 106 may include a seal 118, e.g., at or proximate a proximal end of handle 106. Seal 118 may define an opening configured to receive a shaft 122 of medical instrument 104. Seal 118 may be configured to form a fluid-tight seal against shaft 122. For example, seal 118 may include a flexible membrane proximate, e.g., around, the opening that permits the opening to accommodate shaft 122 while maintaining snug contact with shaft 122 to establish a fluid-tight seal. The flexible membrane may be movable (e.g., radially and/or longitudinally) relative to other parts or portions of the seal. Features of exemplary seals that may be used with medical device 102 are discussed below. In these examples, the seal may comprise one or more biocompatible material, e.g., one or more biocompatible polymers. An entirety of the seal may comprise the same material(s) (optionally with different durometers for different parts of the seal) or may comprise different materials (e.g., a first portion of the seal comprising a different material than a second portion of the seal).

During use, a distal portion of medical device 102 may be inserted an orifice of a subject. For example, sheath 108 may be at least partially inserted into an orifice of the subject. Shaft 122 of medical instrument 104 may be inserted through seal 118 of medical device 102 and advanced through handle 106 and lumen 110 of sheath 108. A fluid-tight seal may be formed between seal 118 and shaft 122 of medical instrument 104. Medical system 100 may be navigated to a target site of the subject before or after inserting medical instrument 103 into seal 118. In an exemplary procedure, port 114 of medical device 102 may be coupled to a vacuum source used to provide suction through lumen 110, e.g., through a channel between the inner surface of sheath 108 and the outer surface of shaft 122 of medical instrument 104. Additionally or alternatively, medical instrument 104 may be used to provide irrigation and/or deliver a tool to the target site through a working channel of medical instrument 104. In some aspects, distal tip 124 of medical device 102 may be proximal of distal opening 110D of sheath 108 during use. In other aspects, distal tip 124 of medical device 102 may be distal of distal opening 110D of sheath 108 during use.

While medical device 102 is used to apply suction via port 114, fluid(s) and/or other material(s) of target site may travel proximally through sheath 108 and removed from the subject via port 114. The fluid-tight seal formed between seal 118 and shaft 122 of medical instrument 104 may help to maintain the desired vacuum pressure within medical device 102. Pressure may be adjusted using opening 116 as discussed above.

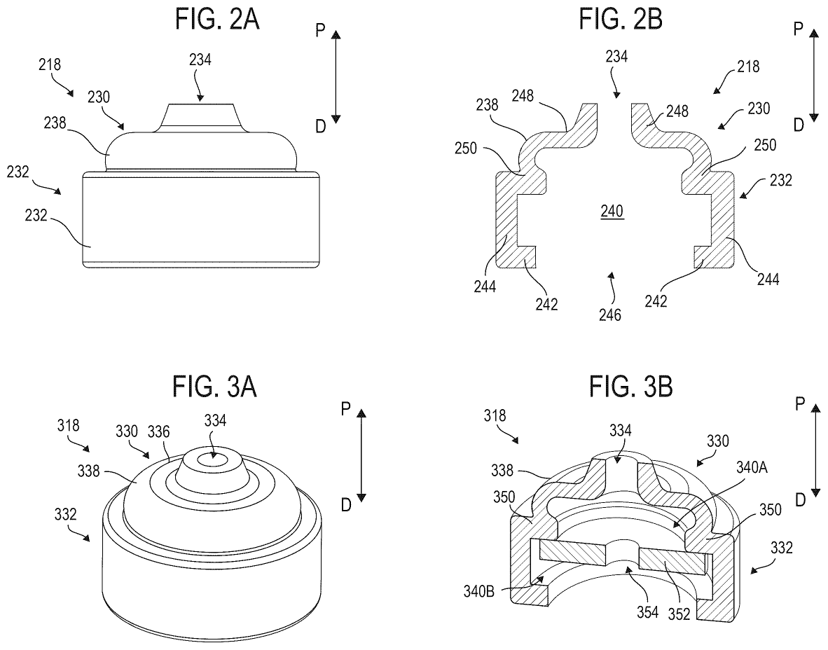

FIGS. 2A and 2B illustrate an exemplary seal 218 configured for use with a medical device, e.g., medical device 102 of medical system 100. For example, FIG. 2A depicts a side view of seal 218, and FIG. 2B depicts a vertical cross-section of seal 218. Seal 218 may be at a proximal end of handle 106 of medical device 102. In some aspects, seal 218 may be coupled to or integrally formed with handle 106. For example, seal 218 may be configured to form a fluid-tight seal against a medical instrument inserted through an opening of seal 218 (e.g., against shaft 122 of medical instrument 104, shown in FIG. 1).

Seal 218 may comprise a first portion 230 and a second portion 232. First portion 230 and second portion 232 may be integrally formed or may be separate components coupled together. First portion 230 and second portion 232 may each comprise a biocompatible material, and may comprise the same or different materials. Exemplary materials useful for seal 218 include, for example, polymers such as medical-grade polyurethane, silicone, polyvinyl chloride, rubber, and other biocompatible polymers. First portion 230 and second portion 232 may comprise the same material having different durometers. For example, first portion 230 may comprise a material having a lower durometer as compared to the material of second portion 232.

First portion 230 may define a proximal opening 234. For example, the material of first portion 230 defining proximal opening 234 may serve as a flexible membrane at or adjacent to a proximal-most end of seal 218. Proximal opening 234 may have a cross-sectional size (e.g., diameter) suitable to receive a shaft of a medical instrument such as a scope, e.g., shaft 122 of medical instrument. In these aspects, the material of first portion 230 serving as the membrane may be sufficiently flexible and elastic to allow proximal opening 234 to receive the shaft and form a fluid-tight seal against the shaft. The membrane may be part of first portion 230 proximate the proximal opening 234 that is movable relative to second portion 232 in response to movement of the shaft extending through proximal opening 234. This flexibility and/or elasticity of the membrane may help to maintain contact between seal 218 and the shaft and help to maintain integrity of the fluid-tight seal. For example, a fold 238 of first portion 230 may stretch or contract longitudinally in a proximal-to-distal direction relative to second portion 232 to allow movement of the shaft within proximal opening 234. In some examples, proximal opening 234 may be tapered in a proximal-to-distal direction or vice versa, for example, to assist with a tight fit against the shaft.

Shown more clearly in FIG. 2B, first portion 230 and second portion 232 may together define an internal cavity 240 in fluid communication with proximal opening 234 and a distal opening 246. When seal 218 is a separate component from a handle of a medical device (e.g., rather than an integral part of the handle), second portion 232 may have one or more features to receive and couple to (e.g., fixedly couple to) the handle of the medical device. For example, a wall 244 of second portion 232 may include a lip 242 configured to receive the proximal end of a handle of a medical device. When seal 218 is integral with handle, lip 242 may be integral with walls of handle 106 distal to seal 218.

Walls of seal 218 may be uniform or may vary in thickness. For example, a portion of a wall 248 at or near proximal opening 234 may be thicker as compared to wall 244 of second portion 232. Similarly, a wall junction 250 between first portion 230 and second portion 232 may be thicker than wall 244 and/or wall 248. The thicker portions may assist in providing support to seal 218, e.g., in order to maintain a fluid-tight connection.

FIGS. 3A and 3B illustrate another exemplary seal 318 useful for medical devices and systems, such as medical device 102 of medical system 100. For example, FIG. 3A illustrates a perspective view of seal 318, and FIG. 3B illustrates a cross-sectional view of seal 318. Seal 318 may have any or all of the characteristics of seals 118 and/or 218. Seal 318 may include a proximal opening 334 configured to receive a shaft of a medical instrument (e.g., shaft 122 of medical instrument 104), e.g., at least a part of first portion 330 being flexible and/or elastic to serve as a membrane. Seal 318 may include a first portion 330 and a second portion 332. First portion 330 (e.g., corresponding to the flexible membrane) may have at least one fold 338.

Internally, seal 318 may have a first cavity 340A in fluid communication with a second cavity 340B. An internal wall 352 with an internal opening 354 may divide first cavity 340A and second cavity 340B. Internal opening 354 may be similarly sized as proximal opening 334. Internal opening 354 may be aligned, e.g., having a center that is coaxial, with proximal opening 334 to receive the shaft of a medical instrument. Internal wall 352 may be configured to form a fluid-tight seal against the shaft at internal opening 354. Optionally, internal opening 354 may be tapered, e.g., in a same direction as proximal opening 334 or in a different direction as proximal opening 334 (when proximal opening 334 is tapered).

Internal wall 352 may be fixed to, or integrally formed with, second portion 332. For example, internal wall 352 may be integral with second portion 332 at or near a wall joint 350 between first portion 330 and second portion 332. In some aspects, the perimeter of internal wall 352 may be spaced apart from a wall of second portion 332 defining second cavity 340B. Internal wall 352 may comprise the same material(s) as first portion 330 and/or second portion 332 or a different material or materials. In some examples, the material of internal wall 352 may have a greater durometer than the material of first portion 330 and/or second portion 332.

During use, proximal opening 334 may form a primary fluid-tight seal against the shaft of a medical device, and internal opening 354 may form a secondary fluid-tight seal against the shaft. First portion 330 including the membrane may be sufficiently flexible to permit maneuverability of the shaft through and within seal 318.

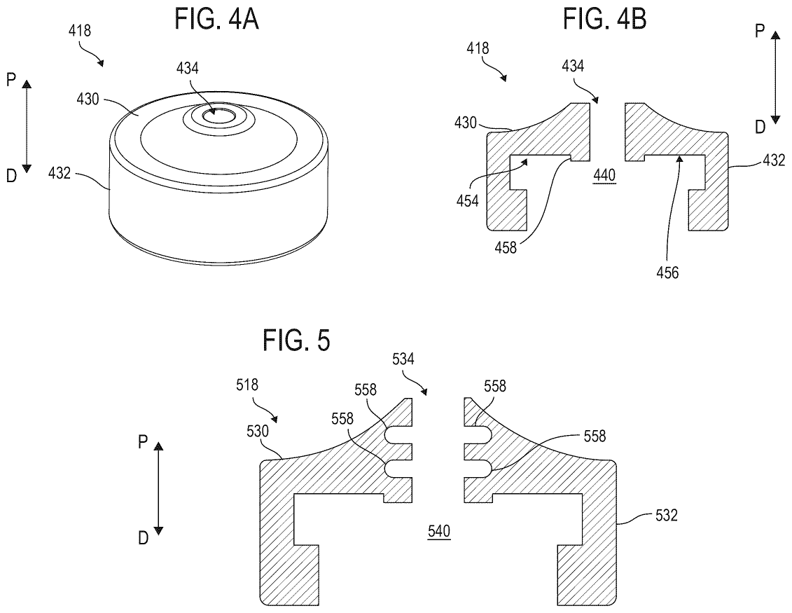

FIGS. 4A and 4B illustrate another exemplary seal 418. For example, FIG. 4A illustrates a perspective view of seal 418, and FIG. 4B illustrates a cross-sectional view of seal 418. Seal 418 may have any or all of the characteristics of seals 118, 218, and/or 318, except as described below. For example, seal 418 may include a proximal opening 434 configured to receive a shaft of a medical instrument (e.g., shaft 122 of medical instrument 104). Proximal opening 434 may be tapered. Seal 418 may include a first portion 430 (including a flexible and/or elastic membrane proximate proximal opening 434) and a second portion 432.

Shown more clearly in FIG. 4B, a wall thickness of first portion 430 may increase at or near proximal opening 434. For example, the wall thickness may decrease towards an outer perimeter of first portion 430. In these aspects, first portion 430 may taper proximally, for example, towards proximal opening 434. The increased thickness at or near proximal opening 434 may provide support or stability for receiving a shaft of a medical instrument. The decreased thickness at or near the outer perimeter of first portion 430 may allow for movement of the shaft within proximal opening 434. In these aspects, a fluid-tight seal with the shaft may be maintained.

Seal 418 may include an internal cavity 440. An inner wall of proximal portion 430 may include a protrusion 458 around proximal opening 434. The portion of the wall that includes protrusion 458 may have a greater thickness, e.g., for increase support when seal 418 receives a shaft of a medical instrument.

FIG. 5 illustrates a cross-sectional view of another exemplary seal 518, which may have any or all of the characteristics of seals 118, 218, 318, and/or 418, except as described below. For example, seal 518 includes a proximal opening 534 configured to receive a shaft of a medical instrument (e.g., shaft 122 of medical instrument 104). Seal 518 may include a first portion 530 (having a flexible and/or elastic membrane proximate to proximal opening 534) and a second portion 532.

Proximal opening 534 (e.g., the narrowed channel within first portion 530 of seal 518) may include one or more annular grooves 558. For example, the wall defining proximal opening 534 may include one groove or two or more grooves 558 spaced apart from one another. Groove(s) 558 may help to decrease friction between seal 518 and a shaft received within proximal opening 534, for example, while maintaining a fluid-tight seal against the shaft.

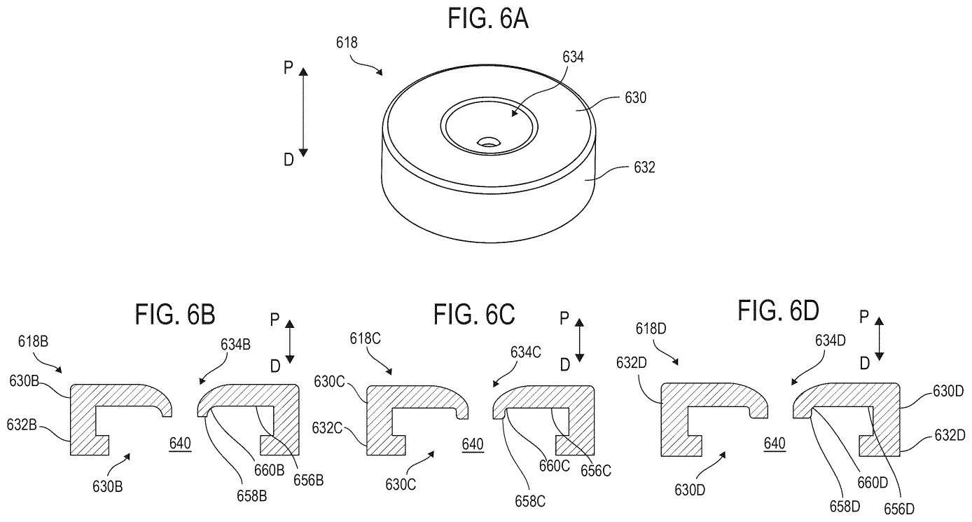

FIG. 6A illustrates a perspective view of another exemplary seal 618. Seal 618 includes a first portion 630 and a second portion 632. Seal 618 may have any or all of the characteristics of seals 118, 218, 318, 418, and/or 518, except as described below. For example, seal 618 may include a proximal opening 634 configured to receive a shaft of a medical instrument (e.g., shaft 122 of medical instrument 104). Part of first portion 630 proximate the proximal opening 634 may serve as a flexible and/or elastic membrane movable relative to second portion 632.

Walls of seal 618 may define an internal cavity. The internal cavity may be configured to couple to a handle of a medical device (e.g., handle 106 of medical device 102) and to receive the medical instrument. Proximal opening 634 may be funnel-shaped. For example, proximal opening 634 may taper such that a proximal end of proximal opening 634 may be larger than a distal end of proximal opening 634. The funnel-shape of proximal opening 634 may be configured to assist with inserting the shaft of the medical instrument through proximal opening 634. The wall of seal 618 may include a protrusion and an edge with an inner fillet, described in further detail below. The thickness of proximal opening 634 may vary, e.g., to provide smooth entry of shaft 122 of medical instrument 104 therethrough. Additionally or alternatively, the thickness of proximal opening 634 may assist in reducing fatigue of seal 618, which may occur due to movement of shaft 122 relative to seal 618.

FIGS. 6B-6D each illustrate a cross-section of several variations on the internal configuration of seal 618, referred to as respective seals 618B-618D, which differ with respect to the protrusion. Each seal 618B, 618C, 618D may be identical externally with seal 618 of FIG. 6A.

Referring to FIG. 6B, seal 618B may include a protrusion 658B extending distally from an internal face 656B of first portion 630B. An edge between protrusion 658B and distal face 656B may include an inner fillet 660B. A thickness of protrusion 658B may decrease, for example, as protrusion 658B extends distally. Inner fillet 660B may be relatively soft, or less sharp, as compared to the other variations illustrated in FIGS. 6C and 6D. Inner fillet 660B may facilitate bending or movement of proximal opening 634B.

Referring to FIG. 6C, seal 618C may include a protrusion 658C extending distally from distal face 656C of first portion 630C. An edge between protrusion 658C and distal face 656C may include an inner fillet 660C. Inner fillet 660C may be sharp (e.g., may form a right angle). In these aspects, a thickness of a wall forming protrusion 658C may be uniform between fillet 660C and a distal end of protrusion 658A. Inner fillet 660C may be relatively sharp, e.g., so as to limit bending or movement of proximal opening 634C. As compared to seal 618B, seal 618C may facilitate less bending or movement of proximal opening 634C.

Referring to FIG. 6D, seal 618D may include a protrusion 658D extending distally from distal face 656D of first portion 630D. An edge between protrusion 658D and distal face 656D may also be sharp, or form a right angle. In these aspects, a thickness of a wall forming protrusion 658D may be uniform between fillet 660D and a distal end of protrusion 658D. A thickness of the wall forming protrusion 658D may be less than or equal to a thickness of the flexible membrane of first portion 630. Inner fillet 660D may be softer than fillet 660C, but sharper than fillet 660B. In some aspects, seal 618D may have a greater thickness at a curvature that defines proximal opening 634D, for example, as compared to inner fillet 660B and fillet 660C. Fillet 660D and the greater thickness of the curvature of proximal opening 634D may result in proximal opening 634D having a greater stiffness, as compared to seal 618B and seal 618C. Accordingly, as compared to seal 618B or seal 618C, seal 618D may facilitate less bending or movement of proximal opening 634D.

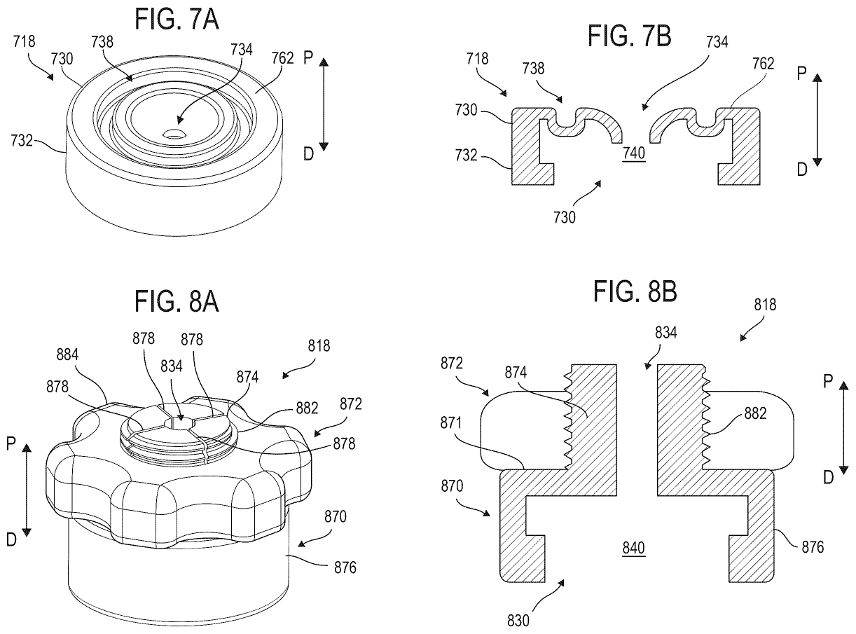

FIGS. 7A and 7B illustrate another exemplary seal 718. For example, FIG. 7A illustrates a perspective view of seal 718, and FIG. 7B illustrates a cross-sectional view of seal 718. Seal 718 may have any or all of the characteristics of seals 118, 218, 318, 418, 518, and/or 618 (including 618B-618D), except as described below. For example, seal 718 may include a proximal opening 734 configured to receive a shaft of a medical instrument (e.g., shaft 122 of medical instrument 104). Proximal opening 734 may be configured to form a fluid-tight seal against the shaft. Proximal opening 734 may be tapered. For example, a cross-sectional dimension (e.g., diameter) of a proximal end of proximal opening 734 may be greater than a diameter of a distal end of proximal opening 734. Seal 718 may include a first portion 730 (including a flexible and/or elastic membrane proximate the proximal opening 734) and a second portion 732. Seal 718 may define an internal cavity 740.

First portion 730 may include at least one fold 738. The fold(s) 738 may define an annular groove, for example, disposed on a proximal face 762 of proximal portion 730. The fold(s) 738 may be configured to expand and contract, for example, as the shaft of the medical instrument is maneuvered within proximal opening 734. In these aspects, a fluid-tight seal may be maintained between the shaft and seal 718.

FIGS. 8A and 8B illustrate another exemplary seal 818 having a first portion 870 and a second portion 872. For example, FIG. 8A illustrates a perspective view of seal 818, and FIG. 8B illustrates a cross-sectional view of seal 818. Seal 818 may have any or all of the characteristics of seals 118, 218, 318, 418, 518, 618 (including 618B-618D), and/or 718 except as described below. For example, seal 818 may include a proximal opening 834 configured to receive a shaft of a medical instrument (e.g., shaft 122). Proximal opening 834 (and part of first portion 870 proximate proximal opening 834 serving as a membrane) may be configured to form a fluid-tight seal against the shaft. A size (e.g., diameter) of proximal opening 834 may be adjustable. For example, the size of proximal opening 834 may change as second portion 872 is moved relative to first portion 870 as discussed further below.

Proximal opening 834 may be tapered. For example, a diameter of a proximal end of proximal opening 834 may be greater than a distal end of proximal opening 834. First portion 870 of seal 818 may include a proximal portion 874 and a distal portion 876. First portion 870 may define an internal cavity 840. Proximal portion 874 of first portion 870 may include a plurality of slits 878 that define proximal opening 834 and serve as a flexible membrane adapted to provide a fluid-tight seal with a shaft of a medical instrument. Slits 878 may extend an entire length of proximal portion 874. Slits 878 may be evenly spaced. Slits 878 may divide proximal portion 874 into a plurality of flexible walls. Proximal portion 874 may include a plurality of threads 882 complementary to a plurality of threads of second portion 872.

Second portion 872 may include gripping surface features. For example, second portion 872 may be configured as a knob grippable by the hand of a user. For example, second portion 872 may include a series of grips 884 to assist a user with relative rotation movement between first portion 870 and second portion 872 (e.g., rotating second portion 872 relative to first portion 870). In some aspects, first portion 870 may be removably coupled to second portion 872.

With relative rotation between first portion 870 and second portion 872, a cross-sectional dimension (e.g., diameter) of proximal opening 834 may change. For example, with second portion 872 in a distalmost position, a proximal end of proximal opening 834 may have a larger diameter as compared to a distal end of proximal opening 834. As second portion 872 moves proximally or is in a proximal-most position, a proximal end of proximal opening 834 may have a same or smaller diameter as compared to a distal end of proximal opening 834. For example, the flexible walls forming the membrane of first portion 870 may be forced inwards (e.g., towards a longitudinal axis of seal 818) as second portion 872 moves proximally or is in a proximal-most position such that the diameter decreases. In some aspects, second portion 872 may serve as a locking mechanism. For example, with relative rotation between first portion 870 and second portion 872 such that a diameter of proximal opening 834 is reduced, translation of shaft 122 distally or proximally within proximal opening 834 may be prevented.

While principles of this disclosure are described herein with reference to illustrative examples, it should be understood that the disclosure is not limited thereto. Those having ordinary skill in the art and access to the teachings provided herein will recognize additional modifications, embodiments, and substitution of equivalents all fall within the scope of the features described herein. Accordingly, the claimed features are not to be considered as limited by the foregoing description.

What is claimed is:

1. A medical device, comprising:
a handle having a proximal end that includes a seal, the seal comprising:
a first portion defining a proximal opening configured to receive a shaft of a medical instrument, at least part of the first portion proximate the proximal opening being a flexible membrane; and
a second portion integral with or coupled to the first portion;
wherein the flexible membrane is movable relative to the second portion to form and maintain a fluid-tight seal with the shaft.

2. The medical device of claim 1, wherein the flexible membrane of the seal is movable relative to the second portion in a radial direction.

3. The medical device of claim 1, wherein the flexible membrane of the seal is movable relative to the second portion in a longitudinal direction.

4. The medical device of claim 1, wherein the proximal opening of the seal is distal to a proximal-most end of the seal.

5. The medical device of claim 1, wherein the proximal opening of the seal is proximal to a proximal-most end of the seal.

6. The medical device of claim 1, wherein the proximal opening of the seal is tapered.

7. The medical device of claim 1, wherein the flexible membrane of the seal includes at least one fold.

8. The medical device of claim 1, wherein the seal includes a cavity in fluid communication with the proximal opening.

9. The medical device of claim 1, wherein a wall of the first portion that defines the proximal opening of the seal includes at least one annular groove.

10. The medical device of claim 1, wherein the seal includes an internal wall between the first portion and the second portion, the internal wall including an internal opening aligned with the proximal opening.

11. The medical device of claim 1, wherein a wall of the seal includes a protrusion extending towards an internal cavity of the seal.

12. The medical device of claim 1, wherein the second portion of the seal is integral with the first portion of the seal.

13. The medical device of claim 1, wherein a wall of the first portion of the seal varies in thickness.

14. The medical device of claim 1, wherein the first portion of the seal has a tapered outer surface.

15. The medical device of claim 1, wherein the seal is fixedly coupled to the handle.

16. A medical device, comprising:
a seal comprising a biocompatible polymer, wherein the seal includes:
a first portion defining a proximal opening configured to receive a shaft of a medical instrument, at least part of the first portion proximate the proximal opening being a flexible membrane; and
a second portion integral with the first portion;
wherein the flexible membrane is movable relative to the second portion to form and maintain a fluid-tight seal with the shaft of the medical instrument.

17. The medical device of claim 16, wherein the flexible membrane is movable relative to the second portion in a radial direction and in a longitudinal direction.

18. A medical device, comprising:
a seal that includes:
a first portion defining a proximal opening configured to receive a shaft of a medical instrument, at least part of the first portion proximate the proximal opening being a flexible membrane; and
a second portion coupled to the first portion;
wherein relative rotation between the first portion and the second portion moves the flexible membrane to thereby increase or decrease a size of the proximal opening and to form and maintain a fluid-tight seal with the shaft of the medical instrument.

19. The medical device of claim 18, wherein the flexible membrane of the seal includes a plurality of slits extending radially outward relative to a longitudinal axis of the seal.

20. The medical device of claim 18, wherein the seal is integrally formed with or fixedly coupled to a handle of the medical device.
